(19) United States
(12) Patent Application Publication
(10) Pub. No.: US 2004/0253200 A1
(43) Pub. Date: Dec. 16, 2004

(54) Capsules

(76) Inventors: Hifzur Rahman Ansari, Old Tappan, NJ (US); Janet Gloria Finnerty, Milford, PA (US); Marcus James Goodall, Kent (GB); Jeremy Nicholas Ness, Kent (GB); Berbara Helen Potts, Milford, PA (US)

Correspondence Address: MORGAN LEWIS & BOCKIUS LLP, 1111 PENNSYLVANIA AVENUE NW, WASHINGTON, DC, 20004, US

(21) Appl. No.: 10/481,957

(22) PCT Filed: Jun. 18, 2002
(86) PCT No.: PCT/GB02/02783

(30) Foreign Application Priority Data
Jun. 28, 2001 (GB) 0115761.9

(51) Int. Cl. A61L 9/015

(52) U.S. Cl. 424/76.2

(57) ABSTRACT

The present invention relates to a method of preventing and/or reducing malodours in an environment, comprising use of an encapsulated perfume, the perfume being encapsulated in capsules comprising perfume in an amount greater than 4% by weight of capsule, and an anionic gum, the method comprising the step of placing the capsules in gaseous contact with the environment, such that perfume released from the capsules prevents and/or reduces malodour in the environment. The capsules typically demonstrate efficient and consistent perfume release over an extended period of time e.g. up to about 85 days and conveniently provide a visual indication of expiration by a change in colour an size during use until a defined end point, when capsule life is exhausted. Also disclosed is a capsule including an emulsifying agent selected from non-ionic surfactants and/or anionic surfactants.

### FIELD OF THE INVENTION

The present invention concerns capsules and their use to prevent and/or reduce malodours in an environment, particularly an enclosed environment. The term “enclosed environment” is used herein to encompass an environment which is permanently enclosed such as a purpose-built air-conditioned office block, an environment which is substantially enclosed such as the underground, metro etc. or an environment which is normally enclosed such as an environment which is openable to external conditions e.g. via a window or door. Thus, the invention finds particular application in domestic or personal environments, but not exclusively so, such as office areas, rooms in buildings e.g. a bedroom or bathroom of a dwelling, or the interior of cars or buses, etc. The invention also concerns novel capsules and products incorporating such capsules.

### BACKGROUND TO THE INVENTION

The problems of preventing and/or reducing malodours in enclosed environments are well known. Such environments have a tendency to become less acceptable to their users, depending upon the type and extent of use, because of possible decreased oxygen availability and the presence of malodorants in the atmosphere. This particularly applies to enclosed spaces where people are present, or pass through, and where environmental factors increase the concentration of malodorants in the atmosphere (for example odours from food or domestic animals). The use of perfumes to reduce the perception of malodours in various environments has been known for many years. Consequently, numerous products employing perfumes have been developed for malodour eradication in domestic or personal environments. An example of such a product is an air freshener in the form of e.g. an aerosol or pump spray, gel etc.

Perfume encapsulation is a well known technique for presenting perfumes, which is typically used to provide perfume stability and/or to extend the lifetime of a perfume. Perfume encapsulates typically find particular application in products for skin care, hair care and cosmetics, personal washing and laundry and textile or fabric softening. For example, WO 99/24159 describes hydrated encapsulates of active material, comprising an emulsion or dispersion of active material in an alginate matrix. The encapsulates of WO 99/24159 are disclosed as providing a convenient delivery system for active material, possibly perfume, and find application as ingredients in a range of water-based foodstuffs, confectionery, pharmaceuticals, cosmetic and oral products. The encapsulates are described as particularly suitable for use in toothpastes as a flavour release system. WO 00/46337 discloses liquid detergent compositions comprising greater than 5% by weight of surfactant and an encapsulate comprising greater than 10% by weight of the encapsulate of active material and a cross-linked anionic gum. The document also discloses liquid detergent compositions generally as defined hereinbefore except the encapsulate comprises greater than 0.5% by weight of the encapsulate of fragrance in an anionic gum. The compositions of WO 00/46337 are disclosed as finding particular application in laundry liquid or fabric detergent products, personal washing products and household cleaning products.

A deodorant comprised of gel beads suitable for treating and deodorising pet litter, which gel beads comprise perfume or essential oil, amongst other ingredients, encapsulated within calcium alginate are disclosed in JP 5049362 and JP 5236837. These documents also disclose a method of making the gel beads.

### SUMMARY OF THE INVENTION

In one aspect the present invention provides a method of preventing and/or reducing malodours in an environment, comprising use of an encapsulated perfume, the perfume being encapsulated in capsules comprising perfume in an amount greater than 4% by weight of capsule, and an anionic gum, the method comprising the step of placing the capsules in gaseous contact with the environment, such that perfume released from the capsules prevents and/or reduces malodour in the environment.

Preferably, the method of the invention comprises use of an encapsulated perfume, the perfume being encapsulated in a plurality of capsules, each capsule comprising anionic gum and perfume in an amount greater than 4% by weight of the total weight of the capsule.

The method of the invention finds particular application in an enclosed environment as defined herein above.

For the purposes of the present invention, the expression “preventing malodours” means that perfume released from the capsules which are in gaseous contact with an environment typically suppresses the olfactory detection of malodours as they are produced in the environment.

For the purposes of the present invention, the expression “reducing malodours” means that perfume released from the capsules which are in gaseous contact with an environment exhibiting malodour typically reduces the perceived intensity of the malodour.

The present invention is based on the appreciation that capsules comprising perfume in an amount greater than 4% by weight of capsule and an anionic gum are conveniently a cost effective means of preventing and/or reducing malodours in an environment, particularly an enclosed environment, when the capsules are in gaseous contact with the environment. Generally, low levels of raw materials are required to produce the capsules which may be prepared via a single step process. Moreover, the capsules described herein surprisingly undergo linear weight loss during use. They are also aesthetically pleasing in comparison with other forms of compositions for preventing and/or reducing malodours, e.g. aerosol sprays. Typically, capsules described herein demonstrate efficient and consistent perfume release e.g. having linear perfume release profiles, leading to controlled perfume release over an extended period of time. Further, capsules described herein provide a visual indication of expiration as they change colour and size i.e. typically becoming darker and undergoing shrinkage during use until a defined end point when capsule life is exhausted, thereby conveniently indicating to the user that the capsules need replacing.

In the simplest case, the capsules described herein are typically employed on their own as beads i.e. in the absence of a carrier. Alternatively, the capsules may be dispersed in a liquid, preferably an aqueous solution, to form a liquid and preferably an aqueous slurry.

When performing the method of the invention, the capsules either in the simplest case as beads, or as part of a liquid and preferably aqueous slurry, may be added to a suitable container of any shape or form, which is open to the atmosphere or is fully or partially openable to the atmosphere. Encapsulated perfume from the capsules may be released into the environment preferably by placing a container comprising capsules in the environment or by spraying, for instance as an aerosol or by a pump action, an aqueous slurry of the capsules from a container. Preferably therefore, the capsules are located in the environment. Alternatively, encapsulated perfume from the capsules may be released at a position remote from the environment in which it is desired to reduce and/or prevent malodours and is carried thereto via passive or active transport from the remote location e.g. by general air movement, or by channelling the released perfume to the environment (e.g. via ducting). If the environment exhibits malodour, typically the capsules will reduce the malodour. Alternatively, if an environment is generally free of malodour, the capsules typically prevent the environment exhibiting malodour as malodours are produced.

Without wishing to be bound or limited by theory, it is thought that perfume is released from the capsules by two different mechanisms, depending upon whether the capsules are part of a liquid (and preferably an aqueous slurry) or employed in the simplest case as beads. In the simplest case as beads, the perfume is thought to simply diffuse through the beads to the capsule surface and then evaporate therefrom. However, when the capsules are part of a liquid, it is thought that perfume diffuses from the capsules into the liquid and evaporates directly from the liquid.

The capsules preferably comprise a hydrated, and more preferably a cross-linked, anionic gum. Preferably, the anionic gum forms a cross-linked matrix. In such a form, perfume is dispersed in a matrix of anionic gum.

Thus, in a further and preferred aspect the invention provides a method of preventing and/or reducing malodours in an environment, comprising use of an encapsulated perfume, the perfume being encapsulated in hydrated cross-linked anionic gum matrix capsules comprising perfume in an amount greater than 4% by weight of capsule, the method comprising the step of placing the capsules in gaseous contact with the environment, such that perfume released from the capsules prevents and/or reduces malodour in the environment.

Alternatively, the capsules may be wall or shell capsules comprising a generally spherical hollow shell or wall of anionic gum within which perfume is contained.

The anionic gum is typically present in an amount of up to about 5% by weight of capsule. Good results have been obtained at lower levels, e.g. constituting about 1% or less by weight of capsule.

By “anionic gum” is meant water-soluble polysaccharides, exuded or extracted from land or marine plants and/or their seeds. The exuded or extracted materials result from normal metabolic processes and are usually in the form of salts of acidic polysaccharides. Modified gums are also included within this definition, which use cellulose as a starting material wherein the hydroxyl groups have been derivatized by a reaction which replaces the hydrogen atoms with a simple or more complex group. Suitable examples of anionic gums for the capsules herein include alginates, carrageenan, gellan gum, carboxyl methyl cellulose and xanthan gum. These gums may be cross-linked as described in WO 00/46337. Mixtures of two or more anionic gums may also be employed, which can result in improved capsule stability. It is preferred to use an alginate anionic gum.

The preferred alginate is conveniently in the form of a sodium salt, and is preferably an alginate having a high percentage of polyguluronic acid units (known as high G alginates) as these produce capsules with a strong structure which maintain the rigidity of the capsules during use. Suitable alginates are commercially available, and include, for example, the high G alginate, Manugel DMB (Manugel is a Trade Mark) supplied by ISP Alginates.

Typically, suitable capsules have a diameter in the range 0.5 to 5.0 mm, preferably 1.0 to 3.0 mm.

The perfume is preferably in the form of an emulsion or dispersion in the anionic gum.

The term “perfume” is used in this specification to mean any odoriferous material generally (but not necessarily) having an odour that is considered pleasant or attractive, or any material which acts as a malodour counteractant, or both.

The perfume may be present in relatively large amounts, typically constituting up to 60% by weight of the total weight of capsule. The perfume is preferably present in an amount in the range from 10 to 50%, more preferably 15 to 40%, and even more preferably 25 to 30% by weight of capsule. By the term “% by weight of capsule” is meant relative to the weight of all capsule ingredients, including water etc.

As is well known, a perfume normally consists of a mixture of several perfumery materials, each of which has an odour or fragrance. The number of perfumery materials in a perfume is typically 10 or more. The range of fragrant materials used in perfumery is very wide; the materials come from a variety of chemical classes, but in general are water-insoluble oils. In many instances, the molecular weight of a perfumery material is in excess of 150, but does not exceed 300.

The perfumes used in the present invention can be mixtures of conventional perfumery materials. Such materials are, for example, natural products such as extracts, essential oils, absolutes, resinoids, resins, concretes etc., but also synthetic materials such as hydrocarbons, alcohols, aldehydes, ketones, ethers, acids, esters, acetals, ketals, nitrites, etc., including saturated and unsaturated compounds, aliphatic, carbocyclic, and heterocyclic compounds.

Such perfumery materials are mentioned, for example, in S. Arctander, Perfume and Flavor Chemicals (Montclair, N.J., 1969), in S. Arctander, Perfume and Flavor Materials of Natural Origin (Elizabeth, N.J., 1960) and in “Flavour and Fragrance Materials—1991”, Allured Publishing Co. Wheaton, Ill. USA.

Examples of perfumery materials which may be used include: acetyl cedrene, 4-acetoxy-3-pentyltetrahydropyran, 4-acetyl-6-t-butyl-1,1-dimetylindane, available under the trademark “CELESTOLIDE”, 5-acetyl-1,1,2,3,3,6-hexamethylindane, available under the trademark “PHANTOLIDE”, 6-acetyl-1-isopropyl-2,3,3,5-tetramethylindane, available under the trademark “TRASEOLIDE”, alpha-n-amylcinnamic aldehyde, amyl salicylate, aubepine, aubepine nitrile, aurantion, 2-t-butylcyclohexyl acetate, 2-t-butylcyclohexanol, 3-(p-t-butylphenyl)propanal, 4-t-butylcyclohexyl acetate, 4-t-butyl-3,5-dinitro-2,6-dimethyl acetophenone, 4-t-butylcyclohexanol, benzoin siam resinoids, benzyl benzoate, benzyl acetate, benzyl propionate, benzyl salicylate, benzyl isoamyl ether, benzyl alcohol, bergamot oil, bornyl acetate, butyl salicylate, carvacrol, cedar atlas oil, cedryl methyl ether, cedryl acetate, cinnamic alcohol, cinnamyl propionate, cis-3-hexenol, cis-3-hexenyl salicylate, citronella oil, citronellol, citronellonitrile, citronellyl acetate, citronellyloxyacetaldehyde, cloveleaf oil, coumarin, 9-decen-1-ol, n-decanal, n-dodecanal, decanol, decyl acetate, diethyl phthalate, dihydromyrcenol, dihyromyrcenyl formate, dihydromyrcenyl acetate, dihydroterpinyl acetate, dimethylbenzyl carbinyl acetate, dimethylbenzylcarbinol, dimethylheptanol, dimethyloctanol, dimyrcetol, diphenyl oxide, ethyl naphthyl ether, ethyl vanillin, ethylene brassylate, eugenol, florocyclene, geraniol, geranium oil, geranonitrile, geranyl nitrile, geranyl acetate, 1,1,2,4,4,7-hexamethyl-6-acetyl-1,2,3,4-tetrahydronaphthalene, available under the trademark “TONALID”, 1,3,4,6,7,8-hexahydro-4,6,6,7,8,8-hexamethylcyclopenta-2-benzopyran, available under the trade mark “GALAXOLIDE”, 2-n-heptylcyclopentanone, 3a,4,5,6,7,7a-hexahydro-4,7-methano-1(3)H-inden-6-ylpropionate, available under the trademark “FLOROCYCLENE”, 3a-4,5,6,7,7a-hexahydro-4,7-methano-1(3)H-inden-6-ylacetate, available under the trademark “JASMACYCLENE”, 4-(4′-hydroxy4′-methylpentyl)-3-cyclohexenecarbaldehyde, alpha-hexylcinammic aldehyde, heliotropin, Hercolyn D which is a trade mark of Hercules Inc, and is a mixture of dihydro and tetrahydro methyl abietate, hexyl aldone, hexyl cinnamic aldehyde, hexyl salicylate, hydroxycitronellal, i-nonyl formate, 3-isocamphylcyclohexanol, 4-isopropylcyclohexanol, 4-isopropylcyclohexyl methanol, indole, ionones, irones, isoamyl salicylate, isoborneol, isobornyl acetate, isobutyl salicylate, isobutylbenzoate, isobutylphenyl acetate, isoeugenol, isolongifolanone, isomethyl ionones, isononanol, isononyl acetate, isopulegol, lavandin oil, lemongrass oil, linalool, linalyl acetate, methyl beta orcinyl carboxylate (LRG 201), 1-menthol, 2-methyl-3-(p-isopropylphenyl)propanal, 2-methyl-3-(p-t-butylphenyl)propanal, 3-methyl-2-pentyl-cyclopentanone, 3-methyl-5-phenyl-pentanol, alpha and beta methyl naphthyl ketones, methyl ionones, methyl dihydrojasmonate, methyl naphthyl ether, methyl 4-propyl phenyl ether, Mousse de chene Yugo, myrtenol, neroli oil, nonanediol-1,3-diacetate, nonanol, nonanolide-1,4, nopol acetate, 1,2,3,4,5,6,7,8-octahydro-2,3,8,8-tetramethyl-2-acetyl-naphthalene, available under the trademark “ISO-E-SUPER”, octanol, Oppoponax resinoid, orange oil, p-t-amylcyclohexanone, p-t-butylmethylhydrocinnamic aldehyde, 2-phenylethanol, 2-phenylethyl acetate, 2-phenylpropanol, 3-phenylpropanol, para-menthan-7-ol, para-t-butylphenyl methyl ether, patchouli oil, pelargene, petitgrain oil, phenoxyethyl isobutyrate, phenylacetaldehyde diethyl acetal, phenylacetaldehyde dimethyl acetal, phenylethyl n-butyl ether, phenylethyl isoamyl ether, phenylethylphenyl acetate, pimento leaf oil, rose-d-oxide, Sandalone, styrallyl acetate, 3,3,5-trimethyl hexyl acetate, 3,5,5-trimethylcyclohexanol, terpineol, terpinyl acetate, tetrahydrogeraniol, tetrahydrolinalool, tetrahydromuguol, tetrahydromyrcenol, thyme oil, trichloromethylphenylcarbinyl acetate, tricyclodecenyl acetate, tricylodecenyl propionate, 10-undecen-1-al, gamma undecalactone, 10-undecen-1-ol, undecanol, vanillin, vetiverol, vetiveryl acetate, vetyvert oil, acetate and propionate esters of alcohol in the list above, aromatic nitromusk fragrances, indane musk fragrances, isochroman musk fragrances, macrocyclic ketones, macrolactone musk fragrances, and tetralin musk fragrances.

Perfumes which are used in this invention may, if desired, have deodorant properties, for example, as disclosed in U.S. Pat. No. 4,303,679, U.S. Pat. No. 4,663,068 and EP-A-545556.

The weight ratio of perfume to anionic gum is preferably in the range 5:1 to 300:1, more preferably 10:1 to 100:1, and most preferably 20:1 to 60:1. The use of high weight ratios of perfume to anionic gum produces cost-effective capsules with good release of perfume over an extended period of time, thus providing an additional environmental benefit.

It may be preferable to use a perfume that has malodour-counteractant properties. Such perfumes function to reduce malodour (i.e. reducing the perceived intensity of a malodour) not by masking the malodour (e.g. dominating the malodour with a stronger odour) but by counteracting or neutralising the malodour in a way that reduces perceived malodour intensity without the need for an intense perfume, or a perfume with a pronounced idiosyncratic odour character such as eucalyptus or wintergreen: this counteracting or neutralising effect is thought to involve some sort of blocking interaction, possibly between the chemical reagents involved, or in the nose or brain of a subject, although the mechanism is not understood.

Such perfumes are known to those skilled in the art, e.g. as disclosed in WO 00/37117.

Additionally or alternatively, it may be preferable to use a perfume having an odour that is considered pleasant or attractive. Thus in a further aspect, the invention provides a method of perfuming an environment, comprising use of an encapsulated perfume, the perfume being encapsulated in capsules comprising perfume in an amount greater than 4% by weight of capsule, and an anionic gum, the method comprising the step of placing the capsules in gaseous contact with the environment, such that perfume released from the capsules provides the environment with a pleasant odour.

Capsules preferably comprise in the range from 40 to 90%, more preferably 60 to 80%, and particularly 70 to 75% by weight of capsule of aqueous solvent, usually water, preferably distilled water.

In order to produce capsules, perfume is either solubilised or emulsified in the anionic gum, preferably by the use of an emulsifying agent. Preferably, the perfume and anionic gum form an emulsion.

Suitable emulsifying agents for use in the capsules described herein include one or more of water-soluble polysaccharides, preferably having a molecular weight of greater than 10,000; water-soluble synthetic resins; and surfactants such as anionic surfactants or non-ionic surfactants, and mixtures thereof.

Suitable water-soluble polysaccharides having a molecular weight of greater than 10,000 include for example, hydroxy cellulosic materials, polysaccharides derived from the monosaccharide glucose, such as hydroxyethyl cellulose (HEC), hydroxypropyl cellulose (HPC) and hydroxypropylmethyl cellulose, or other polysaccharides containing different sugar monosaccharides where some of the primary alcohol groups of the polysaccharide have been oxidised to form uronic acid, for example D-glucuronic acid formed by the oxidation of the primary alcohol groups of glucose. Other examples include D-mannuronic acid and D-galacturonic acid. Such polysaccharides and their derivatives include pectins, gum tragacanth, alginates, propylene glycol alginate, gum arabic and gum ghatti. Combinations of different gums can also be used to achieve superior emulsion stability, such as the combination of propylene glycol alginate and pectins. These materials, particularly propylene glycol alginate, have good emulsifying properties and generally result in the production of capsules with good stability.

Additionally or alternatively, the emulsifying agent may comprise a water-soluble synthetic resin such as polyvinyl alcohol which has excellent film-forming, adhesion and emulsifying properties. Superior emulsion stability can be gained by using a grade of polyvinyl alcohol with a low hydrolysis level and low molecular weight.

The water-soluble polysaccharide and water-soluble synthetic resin emulsifying agents, if present, are typically in an amount in the range from 0.2 to 5%, more preferably 0.3 to 2% and particularly 0.5 to 1% by weight of capsule.

Preferably, the emulsifying agent comprises low molecular weight emulsifying agents such as non-ionic or anionic surfactants, and mixtures thereof, to encourage the release of perfume from the finished capsules during use.

Thus, in a further aspect, the invention provides a capsule comprising;

(i) from 0.5 to 20% by weight of the capsule of an emulsifying agent selected from anionic and non-ionic surfactants, or mixtures thereof; and

(ii) greater than 4% by weight of the capsule of perfume in an anionic gum.

Non-limiting examples of anionic surfactants suitable for use in the capsules of the present invention include alkarylsulphonates e.g. sodium dodecylbenzene sulphonate; alcohol sulphates e.g. sodium lauryl sulphate; ether sulphates e.g. sodium lauryl sulphate with 2 ethylene oxide units; paraffin-sulphonates e.g. sodium alkane sulphonates; sarcosinates—e.g. sodium lauryl sarcosinate; and taurates e.g. sodium N-methyl-N-cocoyl-taurate.

Non-limiting examples of non-ionic surfactants suitable for use in the capsules of the present invention include ethoxylated alkylphenol ethers, particularly octyl- and nonylphenyl ethers containing 5-20 ethylene oxide units; ethoxylated sorbitan esters containing 5-30 ethylene oxide units e.g. Tween 20 which is polyoxyethylene (20) sorbitan monolaurate available from ICI—Tween 20 is a Trade Mark; ethoxylated aliphatic C₆-C₂₀ alcohols which may be linear, branched or Guerbet alcohols (branched fatty alcohols with 100% defined branching at the second carbon position e.g. 2-octyl-1-dodecanol) containing 2-30 ethylene oxide units; polyethylene glycol (2-10 ethylene oxide units) mono and diesters of aliphatic C₅-C₁₁ carboxylic acids; ethoxylated castor oil or hydrogenated castor oil derivatives containing 10-60 ethylene oxide units e.g. Cremophor RH40 available from BASF; and mixed propoxylated/ethoxylated C₄-C₁₆ aliphatic alcohols e.g. Eumulgin L available from Henkel—Eumulgin is a Trade Mark.

The selection of a surfactant (or mixture of surfactants) for inclusion in a capsule will be made according to following considerations; the need for cost-effective emulsification and the nature of the perfume used. It is well known in the art that it is necessary to match the surfactant(s) used with the perfume to achieve good results. If a clear or colourless capsule (i.e. one in which the perfume is solubilised) is required, then it is typically advantageous to carefully choose the surfactant or combination of surfactants to achieve this—see the teachings of EP 0 572 080 in this regard.

Capsules are typically prepared in the manner generally taught in WO 00/46337, e.g. by mixing anionic gum, perfume, desirably emulsifying agent and water to produce an aqueous emulsion, and contacting droplets of the emulsion with an aqueous solution of those monovalent or polyvalent metal ions known to form insoluble gels e.g. calcium or zinc e.g. by spraying droplets of the emulsion into calcium chloride solution.

Cationic surfactants are typically unsuitable for use in the capsules described herein as they may interact with the anionic gum, particularly alginate, and possibly other anionic gums in the emulsion, by forming an insoluble complex with the anionic gum, particularly alginate, which precipitates out of the emulsion causing practical difficulties in forming the capsules.

The anionic surfactant or non-ionic surfactant, or mixture thereof, is preferably present in an amount in the range from 0.5 to 15% by weight of the capsule. The amount of surfactant employed will generally depend upon the nature of, and amount at which, the perfume is employed, which amount can be readily determined by a person of ordinary skill in the art.

In a preferred embodiment herein, capsules preferably comprise in addition to an emulsifying agent, one or more water-soluble solvents. The inclusion of water-soluble solvents in capsules herein may improve the clarity of the capsules, if desired, as well as improve the release of perfume from the capsules in-use.

Suitable water-soluble solvents for use herein include alcohols (e.g. ethanol, glycerol) and glycols such as propylene glycol, dipropylene glycol, diethylene glycol monoethyl ether etc., typically present in an amount in the range of from 1 to 20% by weight of the capsule. If such a solvent is used, then a similar or greater level of solvent will generally be required to be added to a cross-linking bath upon preparation of the capsules.

The absence of water-soluble solvents in a capsule may be advantageous, if it desirable to produce capsules which release lower levels of volatile organic chemicals.

Capsules conveniently optionally comprise a colouring agent in the form of a pigment, e.g. titanium oxide for white colour or, oil soluble colour to improve the aesthetic appearance of the capsule e.g. to produce an attractive coloured capsule instead of a clear or colourless capsule. Preferably, the colouring agent is a pigment.

Preferably, capsules according to the invention are utilised as air freshening products. The invention in an even further aspect therefore provides an air freshener comprising capsules in accordance with the invention.

In a further aspect the invention provides the use of capsules comprising greater than 4% by weight of a capsule of perfume in an anionic gum for preventing and/or reducing malodours in an environment.

The invention will be further described, by way of illustration, in the following examples, and with reference to the accompanying drawing, in which:

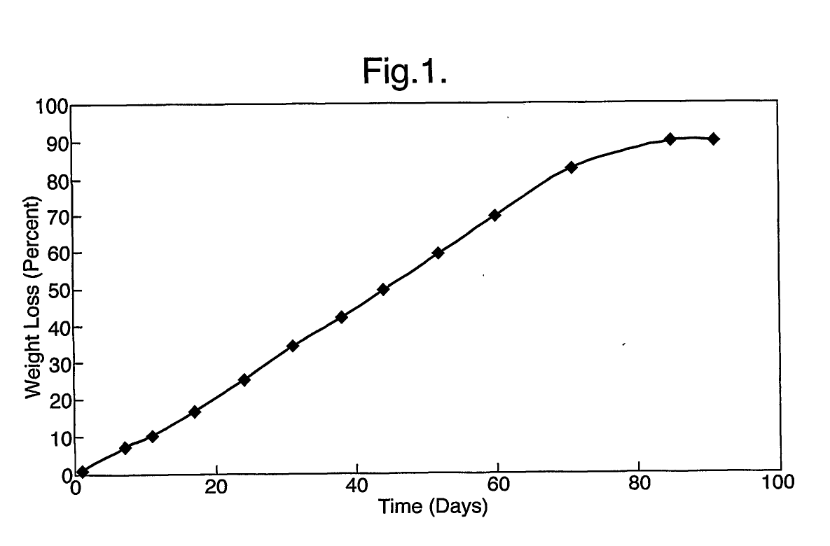

FIG. 1 is a graph illustrating the percentage weight loss from capsules, in the simplest case as beads, over time (in days).

### EXAMPLES

Examples of capsules embodying the invention are as follows:

| % w/w | 1 | 2 | 3 | 4 | 5 | 6 | 7 | 8 |
|---|---|---|---|---|---|---|---|---|
| Distilled Water | 73.4 | 68.8 | 78.8 | 88.8 | 71.39 | 71.39 | 71.39 | 71.39 |
| Sodium algi- | 0.8 | 0.6 | 0.6 | 0.6 | 0.8 | 0.8 | 0.8 | 0.8 |
| nate (Manu- | | | | | | | | |
| gel DMB) | | | | | | | | |
| Propylene gly- | 0.4 | 0.2 | 0.2 | 0.2 | 0.4 | 0.4 | 0.4 | 0.4 |
| col alginate | | | | | | | | |
| (Kalcoloid S) | | | | | | | | |
| Perfume Oil¹ | 25.0 | 15.0 | 10.0 | 5.0 | 25.0 | 10.0 | 15.0 | 20.0 |
| Tween 20 | — | 15.0 | 10.0 | 5.0 | — | — | — | — |
| Gellan gum | — | — | — | — | 0.01 | 0.01 | 0.01 | 0.01 |
| (Kelcogel F) | | | | | | | | |
| Colloidal silica | — | — | — | — | 2.0 | 2.0 | 2.0 | 2.0 |
| (Ludox HS-40) | | | | | | | | |
| Glydant | 0.4 | 0.4 | 0.4 | 0.4 | 0.4 | 0.4 | 0.4 | 0.4 |
| (preservative) | | | | | | | | |
| Dipropylene | — | — | — | — | — | 15.0 | 10.0 | 5.0 |
| glycol | | | | | | | | |
| Colour | q.s. | q.s. | q.s. | q.s. | q.s. | q.s. | q.s. | q.s. |

¹Peach #656-842S commercially available from Quest

Manugel DMB is a Trade Mark and is available from ISP Alginates

Kelcoloid S is a Trade Mark and is available from ISP Alginates

Tween 20 is a Trade Mark and is polyoxyethylene (20) sorbitan monolaurate, available from Uniqema

Kelcogel F is a Trade Mark and is available from ISP Alginates

Ludox HS-40 is a Trade Mark and is available from DuPont

Glydant is a Trade Mark and is available from Lonza

The capsules described above in Examples 1 to 8 were prepared by the method described in Example 1 of WO 00/46337 to produce capsule beads of approximately 2 mm in diameter. Where the capsules included dipropylene glycol, this was also present in the calcium chloride reservoir solution in the same amount.

The percentage weight loss of perfume from capsule beads of Example 3 above was then measured to determine the rate of emission of perfume therefrom.

The percentage weight loss of perfume from the beads was measured by weighing a suitable container on an accurate balance to determine its mass (g). To the container was then added a known quantity (g) of capsule beads. The container with beads was then placed in a room and weighed at appropriate intervals during use, namely after 1, 7, 17, 24, 38, 44, 52, 71 and 85 days, with the net weight of the capsule beads (i.e. not including the weight of the container) calculated and recorded at each interval.

It was found, as illustrated in FIG. 1, that the capsule beads of Example 3 demonstrated linear weight loss over approximately 85 days, after which time the rate of perfume release reached a plateau with approximately 89% of the perfume being emitted therefrom.

1. A method of preventing and/or reducing malodours in an environment, comprising use of an encapsulated perfume, the perfume being encapsulated in capsules comprising perfume in an amount greater than 4% by weight of capsule, and an anionic gum, the method comprising the step of placing the capsules in gaseous contact with the environment, such that perfume released from the capsules prevents and/or reduces malodour in the environment.

2. A method according to claim 1, wherein the environment is an enclosed environment.

3. A method according to claim 1 or 2, wherein the capsules are located in the environment.

4. A method according to any one of the preceding claims, wherein the capsules are added to a container.

5. A method according to any one of the preceding claims, wherein the capsules are dispersed in a liquid to form a slurry.

6. A capsule comprising;
(i) from 0.5 to 20% by weight of the capsule of an emulsifying agent selected from anionic and non-ionic surfactants, or mixtures thereof; and
(ii) greater than 4% by weight of the capsule of perfume in an anionic gum.

7. A capsule according to claim 6, wherein the capsule additionally comprises a water-soluble solvent.

8. A capsule according to claim 6 or 7, wherein the anionic gum of the capsule is hydrated.

9. A capsule according to any one of claims 6, 7 or 8, wherein the anionic gum forms a cross-linked matrix.

10. A capsule according to any one of claims 6 to 9, wherein the anionic gum is present in an amount up to 5%, preferably up to 1% by weight of the capsule.

11. A capsule according to any one of claims 6 to 10, wherein the anionic gum is alginate, carrageenan, gellan gum, carboxymethyl cellulose and/or xanthan gum.

12. A capsule according to claim 11, wherein the anionic gum is alginate.

13. An air freshener comprising capsules in accordance with any one of claims 6 to 12.

14. A method according to any one of claims 1 to 5, wherein the capsules are as specified in any one of claims 6 to 12.

15. Use of capsules comprising greater than 4% by weight of a capsule of perfume in an anionic gum for preventing and/or reducing malodours in an environment.

16. A method of perfuming an environment, comprising use of an encapsulated perfume, the perfume being encapsulated in capsules comprising perfume in an amount greater than 4% by weight of capsule, and an anionic gum, the method comprising the step of placing the capsules in gaseous contact with the environment, such that perfume released from the capsules provides the environment with a pleasant odour.
